(19) United States
(12) Patent Application Publication
(10) Pub. No.: US 2018/0243491 A1
(43) Pub. Date: Aug. 30, 2018

(54) PUMP FOR CONVEYING FLUIDS, AND METHOD FOR DETERMINING A FLOW RATE

(71) Applicant: Berlin Heart GmbH, Berlin (DE)

(72) Inventors: Constantin Wiesener, Potsdam (DE); Marcus Granegger, Zürich (DE); Michael Frischke, Rangsdorf (DE)

(73) Assignee: Berlin Heart GmbH, Berlin (DE)

(21) Appl. No.: 15/756,092

(22) PCT Filed: Aug. 29, 2016
(86) PCT No.: PCT/EP2016/070325

§ 371 Date: Feb. 28, 2018

(30) Foreign Application Priority Data
Aug. 31, 2015 (EP) 15183195.5

(51) Int. Cl. A61M 1/10; A61M 1/12

(52) U.S. Cl. CPC A61M 1/1086; A61M 1/1029; A61M 2205/3368; A61M 2205/3334; A61M 1/122

(57) ABSTRACT

A pump for conveying fluids, in particular a blood pump, comprising a device for determining the through-flow rate. The device for determining the through-flow rate can be advantageously implemented via a device integrated in the pump for supplying or discharging heat energy to or from the conveyed fluid, as well as a device integrated in the pump for determining a temperature, a temperature change or a temperature gradient.

### CROSS-REFERENCE TO RELATED APPLICATIONS

This application is a 371 nationalization of international patent application PCT/EP2016/070325 filed Aug. 29, 2016, which claims priority under 35 USC § 119 to European patent application EP 15183195.5 filed on Aug. 31, 2015, both of which are hereby entirely incorporated by reference.

### TECHNICAL FIELD

The invention lies in the field of electrical engineering and mechanical engineering and can be used particularly advantageously in the field of medical technology.

### BRIEF DESCRIPTION OF THE DRAWINGS

FIG. 1 shows schematically a blood pump with two marked positions for through-flow rate sensors in a longitudinal section;

FIG. 2 shows a pump similar to the embodiment in FIG. 1 in a plan view;

FIG. 3 shows schematically a pump similar to that in FIG. 1 in a longitudinal section, wherein the drive motor is shown in somewhat greater detail;

FIG. 4 shows a pump similar to that in FIG. 3, in which an axial bearing unit is additionally shown in somewhat greater detail;

FIG. 5 shows schematically the construction of an exemplary through-flow rate sensor; and

FIG. 6 shows the arrangement of a heating element and a reference temperature sensor externally on a pump tube.

### DETAILED DESCRIPTION

More specifically, the invention relates to pumps for conveying fluids.

It is often necessary to determine the flow rate within the scope of the conveyance of a fluid by a pump. This is possible for example by the detection of the motor current and a speed of rotation and the known relationships between these parameters and the volume flow rate in a pump.

In some cases an estimation method of this kind is not accurate or reliable enough, in particular if the viscosity changes or there is an additional load torque on the rotor (for example thrombus).

Lastly, in the case of rotary pumps with active magnetic bearings, it is possible, by monitoring the forces in the controlled bearings, to determine a pressure difference across the pump and on this basis to estimate a flow rate under consideration of further detected parameters.

The object of the present innovation is to create a pump for conveying fluids, which pump allows the through-flow rate to be determined in the most reliable manner possible. The object is solved, in particular in the case of a blood pump with a pump tube, by means of a device for determining the through-flow rate, wherein a device integrated in the pump for supplying or discharging heat energy to or from the conveyed fluid and also a device integrated in the pump for determining a temperature, a temperature change, or a temperature gradient are provided.

If heat energy is supplied to or removed from the fluid to be conveyed by means of a temperature element (device for supplying or discharging heat energy), the temperature thus changes as a function of the time that the fluid to be conveyed is in contact with the device for supplying or discharging heat energy, and therefore as a function of the through-flow rate. The temperature or temperature change attained hereby or the temperature gradient attained hereby is measured and used to determine a through-flow rate by means of known reference values or a set of characteristic curves. One embodiment is configured in such a way that a heater and/or other heat sources heat the blood-conducting side of the pump tube by no more than 2 Kelvin in relation to the blood temperature (prior to entry into the blood pump).

The device integrated in the pump for supplying or discharging heat energy can be a temperature element for example, which “produces” or “absorbs” heat (this means of course: there is a conversion between heat energy and another energy form) and which is directly in contact with the conveyed fluid, for example said fluid flows around said device. For example, the device can be a heating wire or a Peltier element.

By means of one or more temperature measurements taken at the fluid that is to be conveyed or in the vicinity of the temperature element, a generated temperature or a temperature difference, a temperature change, or a temperature gradient can be determined. For example, in conjunction with the pump, a first temperature sensor can be provided at the pump inlet, with a temperature element arranged downstream thereof with respect to the conveyance direction of the fluid, and with a further temperature sensor arranged further downstream, for example at the pump outlet.

Similarly to the thermodilution method for measuring cardiac output per minute, the temperature element can be controlled accordingly, so that a reproducible heat input into the fluid is produced. A flow rate through the pump can then be determined from the temperature and progression thereof or the temperature difference and progression between the temperatures measured at the pump inlet and at the pump outlet.

The use of a flowmeter in a pump is known in principle from U.S. Pat. No. 4,465,063, however said flowmeter is installed in that case in a secondary circuit of a fluid (gas) driving the pump and is not in direct contact with the fluid to be conveyed.

One embodiment of the invention provides that a heating element is directly in heat contact with the pump tube and that a device for determining the temperature of the heating element is provided, which device either is integrated in the heating element or is provided as a separate temperature sensor.

By feeding a certain heating capacity, the temperature of the heating element can thus be increased. The temperature increase is transferred to the pump tube, which directly conducts the blood flow. The pump tube and the heating element are cooled by the blood flowing along the wall of the pump tube to an extent dependent on the flow velocity of the blood. The flow velocity of the blood and therefore also the through-flow rate can thus be determined from the temperature increase or the temperature measured in absolute terms. The temperature sensor can be designed in such a way that the heating element is formed as an electric resistance heater and the resistor is formed as a temperature-dependent electric resistor. The temperature can then be determined from the voltage drop at the heating resistor. However, a thermocouple can also be connected as a temperature sensor to the heating element/heating resistor or can be arranged in the direct vicinity thereof.

A further embodiment can provide that a reference temperature sensor is arranged on the pump tube at a distance from the heating element, in particular at a distance of at least 2 mm, more particularly at a distance of at least 4 mm, and is in direct heat contact therewith.

The temperature of the blood can be determined directly in the blood or by means of the temperature of the pump tube using a reference temperature sensor of this kind. The temperature measurement by means of the reference temperature sensor is taken at a point at which the blood temperature is uninfluenced to the greatest possible extent by the heating element. The blood temperature can thus be used as a correction variable when determining the flow velocity.

It can additionally be provided that the heating element and the reference temperature sensor are arranged on the pump tube substantially in the same axial position with respect to the longitudinal axis of the pump tube and at a distance from one another in the peripheral direction.

It can thus be ensured in a simple way that the reference temperature sensor measures the blood temperature independently of the heating effect of the heating element. The elements (heating element and reference temperature sensor) can thus also be arranged in a space-saving manner.

By means of an arrangement of this kind, the reference temperature measurement is also independent of the through-flow rate or flow velocity.

A further embodiment can provide that the heating element and the device for determining the temperature of the heating element and/or the reference temperature sensor are arranged on the outer side of the pump tube. The sensors and the heating element are thus on the one hand easily accessible and on the other hand do not interfere with the blood flow in the pump tube. The pump tube itself is designed in such a way in respect of its shaping, wall thickness and material selection that on the one hand it dissipates the heat well enough to the blood, but on the other hand a temperature increase does not primarily propagate along the pump tube in the material thereof. A hot spot is formed at the pump tube by the operation of the heating element, which hot spot primarily exchanges heat with the blood flowing past.

It can additionally be provided that the pump tube is made of titanium or a titanium alloy and, at least in a portion in which the heating element is arranged, has in particular a wall thickness between 0.1 mm and 1 mm, more particularly between 0.1 mm and 0.7 mm. Titanium and titanium alloys have a heat conduction coefficient which is optimal for achieving the abovementioned objectives. To this end, the correct choice of the wall thickness of the pump tube is additionally important. Here, the required heat properties have to be balanced in relation to the necessary mechanical stability.

A particular embodiment of the invention provides that a device for supplying heat energy to the conveyed fluid is formed by a drive motor integrated in the pump. The heat energy converted in the drive motor of the pump can be calculated from the motor current and voltage, and therefore it is possible to determine the heat input into the fluid on this basis. A temperature measurement downstream or temperature measurements in the pump upstream and downstream of the point at which the drive motor can transfer heat to the conveyed fluid then make it possible to determine the through-flow rate through the pump under consideration of the motor power.

It can also be provided that a device for supplying heat energy to the conveyed fluid is formed by one or more active, in particular magnetic bearings integrated in the pump. The bearings of a pump rotor can also be considered to be heat sources, the power of which can be determined, for example in the case of controlled magnetic bearings, by a monitoring of the current through the bearing electromagnets. The heat energy input by the bearings can also be used in conjunction with the determined temperature or a determined temperature difference in order to determine the through-flow rate. The sum of the power converted in the bearings and the motor drive power as heat input can also be taken into consideration.

A further embodiment of the invention can provide that a heating or cooling element arranged in the flow of the fluid to be conveyed, in particular in the form of a heating wire or a cooling element, in particular a Peltier element, is arranged in or directly on the housing of the pump. A defined positive or negative heat input can be introduced into the flowing fluid by means of a heating or cooling element of this kind, which can be formed as a heating wire or also as a Peltier element or in another known form, and the temperature change or a temperature gradient can be detected by corresponding sensors close to the temperature element itself or for example upstream and downstream of the heating or cooling element. In this case, the device functions similarly to a heating wire anemometer.

As already explained in part above, in order to implement the invention it can be provided that a device for determining the temperature, a temperature change or a temperature gradient comprises a first and/or a second temperature sensor, wherein, in the flow of the fluid to be conveyed, one of the temperature sensors is arranged upstream and the other temperature sensor is arranged downstream of the device for supplying or discharging heat energy.

Here, it can also be provided for example that a device for determining the temperature, a temperature change or a temperature gradient comprises a first and/or a second temperature sensor, wherein, in the flow of the fluid to be conveyed, one of the temperature sensors is arranged upstream of a pump rotor and the other temperature sensor is arranged downstream of the pump rotor.

Here, the pump rotor can also comprise the drive motor of the pump, so that the heat input by the motor is also taken into consideration, either as a single heat input or additionally to the heat input by a heating element. A cooling element can also be combined with the heat input by the motor, and the total heat balance can be detected by temperature measurements.

It is also conceivable that a device for determining the temperature or a temperature change is arranged directly on the heating or cooling element or is integrated therein. The temperature measurement or the detection of the temperature change or of a gradient is thus performed directly at the point at which the heat input or the discharge of heat occurs, such that other influences can be largely ruled out, and, in the case that the device for determining the temperature or a temperature change is directly integrated in the heating or cooling element, a physical property of the element that changes with the temperature can be used directly to detect the temperature variable, for example when a temperature-dependent resistor or a thermocouple is integrated in the heating or cooling element.

It can also be provided in the case of the invention that a device for determining a temperature change is arranged directly on the heating or cooling element and that a control device for adjusting a temperature, a temperature change or a temperature gradient to a given target value is provided, wherein the heating or cooling power that is to be supplied in order to achieve the target value is controlled. A certain temperature or a certain temperature gradient can thus be attained by a control operation, wherein the through-flow rate can be determined by the influencing variable necessary for the control, for example the supplied energy (for example current). The advantage of this method and this device lies in the fact that the working temperature and the temperature difference relative to the surrounding environment (=blood) remains the same and therefore temperature-dependent variables that influence the determination of the through-flow rate remain excluded.

It can also be provided that a heat-dissipating region and/or a cooling region of a temperature element are/is arranged in the flow of the fluid to be conveyed, and that one or more temperature sensors is/are provided for continuous detection of the temperature of the element and/or of the fluid flowing past. In this case, it is not the temperature development of the fluid to be conveyed that is used, but instead the temperature development of the temperature element, i.e. of the heating or cooling element. This is heated or cooled in principle by an electrical controller, wherein heat is transported to the surrounding medium in a manner dependent on the through-flow rate. A device of this kind allows the temperature element comprising the temperature sensors to be integrated particularly well.

In addition, it can be provided that a temperature-dependent electric resistor or a thermocouple is provided in order to detect the temperature of the temperature element.

The invention also relates to a pump of the above-described type and also to a method for determining the flow rate of a fluid to be conveyed by a pump of the above-described type, wherein the flow rate is determined under consideration of a detected heating or cooling power, temperature, a temperature difference, an electric motor power, and in particular a speed of rotation of the pump and an electric bearing performance.

A particular embodiment of the invention can lie for example in the fact that an exchange region of a temperature element, in particular a Peltier element, connected to the fluid to be conveyed is cooled and heated in alternation, and in the fact that the temperature of the exchange region is measured continuously and the flow velocity is determined on the basis of the temperature changes and/or heating or cooling capacity.

Hereinafter, exemplary embodiments of the invention will be shown on the basis of figures of a drawing and will be explained further below. In the drawing

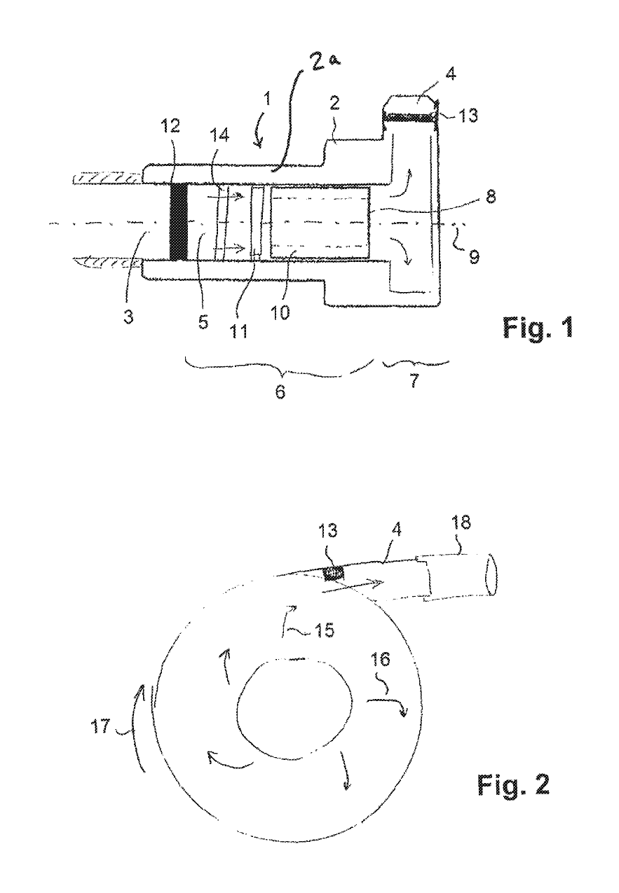

FIG. 1 shows schematically a blood pump with two marked positions for through-flow rate sensors in a longitudinal section,

FIG. 2 shows a pump similar to the embodiment in FIG. 1 in a plan view,

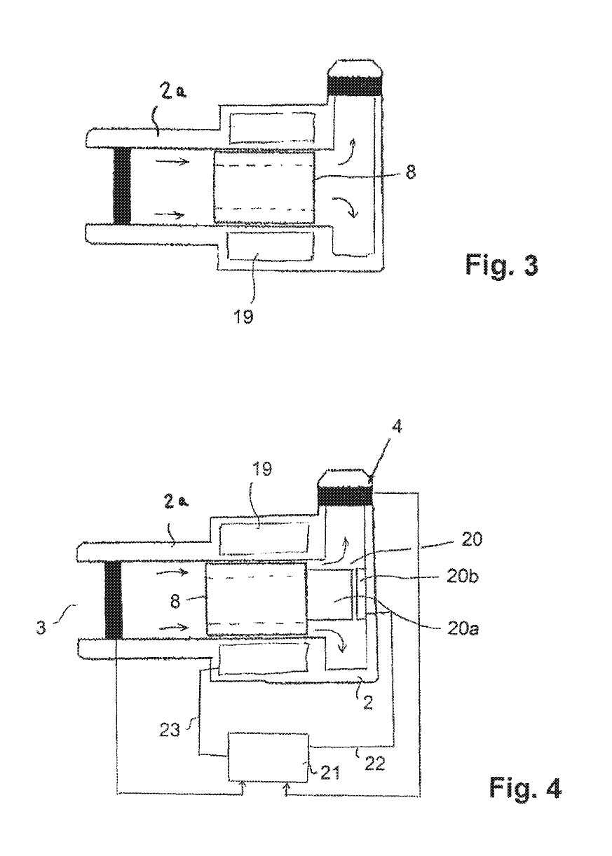

FIG. 3 shows schematically a pump similar to that in FIG. 1 in a longitudinal section, wherein the drive motor is shown in somewhat greater detail,

FIG. 4 shows a pump similar to that in FIG. 3, in which an axial bearing unit is additionally shown in somewhat greater detail,

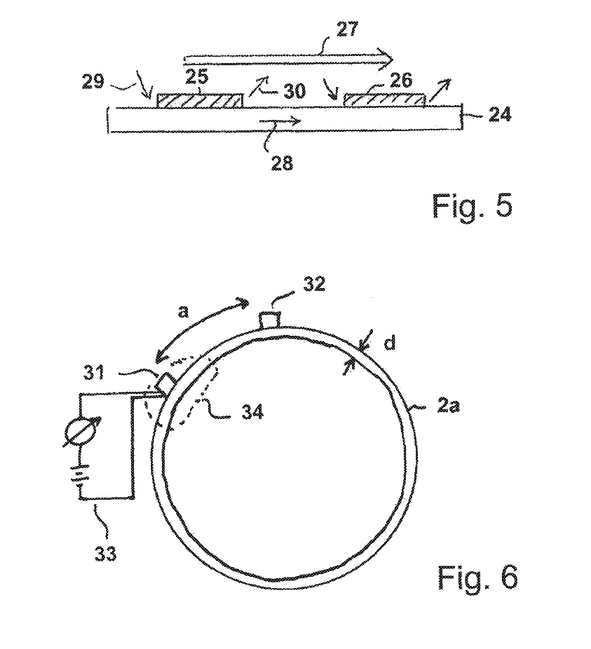

FIG. 5 shows schematically the construction of an exemplary through-flow rate sensor, and

FIG. 6 shows the arrangement of a heating element and a reference temperature sensor externally on a pump tube.

FIG. 1 shows schematically, in a longitudinal section, a pump 1 according to the invention, which pump for example can be a blood pump for use in living patients. A pump of this kind can be implantable or partially implantable. Pumps of this kind, however, can also be used for other medical or non-medical purposes. Particular advantages result from a space-saving integration and/or structural connection of one or more through-flow rate sensors to the pump.

A pump of this kind typically has a pump housing 2 with a pump inlet 3 and a pump outlet 4. A fluid channel 5 is provided between the inlet 3 and the outlet 4, which fluid channel in the shown example runs in a first region 6 in the axial direction and in a second region 7 at least partly in the radial direction. The pump housing 2 has a pump tube 2a at least in part between the inlet 3 and the outlet 4, with the fluid channel 5 running in said pump tube. A rotor 8 is provided within the axial region 6 of the fluid channel 5 and can be driven about the rotation axis 9 and has, in its radial outer region, conveying elements 10 in the form of conveying blades, which are not shown in detail.

The fluid, for example blood, is conveyed from the pump inlet 3 in the axial direction 9 by the rotor 8 with its conveying elements 10, wherein the fluid is additionally swirled and in the second region 7 of the fluid channel 5 is pushed outwardly at least partially in the radial direction and in the peripheral direction, wherein it is conveyed by centrifugal forces to the pump outlet arranged radially outwardly.

It should be noted that the present invention, however, is also applicable to purely axially conveying pumps or pumps having different conveying principles.

In order to determine the through-flow rate of the pump, through-flow rate sensors should be arranged in a region of the fluid channel 5 in which the flow is as homogeneous as possible. To this end, through-flow rate sensors for example should be arranged expediently directly on the pump inlet and/or on the pump outlet 4. However, it is not ruled out that through-flow rate sensors of this kind can also be arranged at other points of the pump where a flow can be locally detected representatively of the total flow through the pump.

A flow guide wheel 11 is shown in FIG. 1 upstream of the rotor 8 in the flow direction and comprises flow-guiding elements, which for example swirl the incoming flow, which improves the conveying capacity of the pump.

One concept forming the basis of the innovation, amongst others, is that the through-flow rate of a pump of this kind can be measured by local input of energy or local dissipation of energy and simultaneous detection of the temperature development, since the development of the temperature with corresponding heat supply or dissipation is determined by the heat transport by the blood flowing past/the fluid flowing past. To this end, a temperature element 14 can be provided within the pump between a first temperature sensor 12 and a second temperature sensor 13, by means of which temperature element a heat energy input into the fluid is possible, for example a heating resistor, which can be controlled electrically. The supplied heat energy can thus be precisely determined, and the temperature difference attained by the heating by means of the temperature element 14 can be determined for example by means of the first and second sensor 12, 13.

However, the heat that is input into the fluid by an electric pump drive can also be taken into consideration in the underlying calculation model, particularly within a pump in which a drive is integrated.

The input energy per unit of time, i.e. the heating capacity, is proportional to the temperature difference between pump inlet and pump outlet in linear approximation. The proportionality factor is determined by a product of the density of the blood, the specific heat capacity, and the flow rate of the blood. On the other hand, the input energy can be calculated from the electrical power of the motor drive of the pump and, if provided, the temperature element 14.

The corresponding equation describing the thermal model of the pump is as follows:

E^{&}ρ·c·Qb(Tᵢₙₗₑₜ−Tₒᵤₜₗₑₜ),

wherein Ė corresponds to the input of heat energy per unit of time, p corresponds to the density of the blood, c corresponds to the specific heat capacity, and Qb corresponds to the flow rate of the blood, Tᵢₙₗₑₜ corresponds to the temperature at the pump inlet, and Tₒᵤₜₗₑₜ corresponds to the temperature at the pump outlet.

FIG. 2, for improved understanding of the pump construction, shows a view in the axial direction 9 of the pump from FIG. 1, wherein the flow direction of the blood in the region 7 of the fluid channel 5 is indicated by arrows 15, 16, wherein the blood is pushed radially outwardly and in the peripheral direction 17 and then flows to the pump outlet 4. There, a cannula 18 is connected, through which the blood is conveyed further.

A stator 19 of a pump drive is schematically shown in FIG. 3 on the pump housing additionally to that shown in FIG. 1, wherein the stator 19 concentrically surrounds the rotor 8 in the pump housing. The rotor 8 contains corresponding magnetic elements, which are used to realise a brushless electric motor. At least some of the heat generated by the current flow in the stator 19 is input into the fluid channel 5.

In FIG. 4 a bearing device 20 is additionally shown, which comprises a first part 20a of the bearing rotating with the rotor 8, and a stationary part 20b of the bearing. The stationary part 20b of the bearing is part of a magnetic circuit, which for example can be closed by parts of the pump housing 2 and the stator 19, wherein a controllable electromagnet is integrated in this magnetic circuit and can control the magnetic forces in the axial direction in order to provide an axial support of the rotor 8. The electrical power necessary for this purpose can also be included in the calculation of the heat supply, i.e. in the calculation of the total heat energy input into the blood flowing through the pump.

To this end, a control unit 21 is schematically shown, which is connected by means of lines 22, 23 to the active, heat-dissipating elements of the pump, i.e. the stator 19 and the bearing device 20, and the input electrical and thus also heat capacity can be determined by means of said control unit. The control unit 21 is additionally connected to the first and second sensor 12, 13, so that it likewise can detect the temperature difference between pump inlet 3 and pump outlet 4. The control unit 21 can comprise a microcontroller, by means of which the blood through-flow rate can be directly determined on the basis of the detected temperature difference and the input power.

The temperature sensors 12, 13 can be held within the pump 1 for example in the region of the pump inlet 3 by means of a supporting star in the fluid channel 5, or a temperature sensor can be arranged in the region of the inlet guide vane 11 before the rotor, and can be fastened to the inlet guide vane 11. A temperature sensor for example can also be fastened to the inner wall of the housing 2 in the region of the pump inlet.

A temperature sensor can be held in the region of the pump outlet 4, likewise in the outlet channel by means of a supporting star. However, the temperature sensor 13 can also be fastened to a wall of the pump outlet channel 4.

The fastening of temperature sensors to a supporting element, in particular to a supporting star, in such a way that the respective temperature elements are distanced from the wall of the pump housing 2 has the advantage that all of the heat produced or absorbed in the temperature element is exchanged with the fluid before heat is conducted to the wall of the housing 2.

The above-described variants for implementing the invention presuppose that the temperature is detected upstream and downstream of the pump rotor or a temperature element.

However, it is also possible to determine the through-flow rate of the fluid by means of a single thermal anemometry element, which can be integrated in a pump. To this end, on the one hand heat is dissipated to the fluid or absorbed from the fluid by means of an element, and on the other hand a temperature change of the temperature element itself is determined at the same time.

To this end, the temperature element can be connected to a sensor, so that for example a heating resistor is coupled to a temperature sensor, for example a thermocouple, or a Peltier element is coupled to a thermocouple, or both the electrical energy in a heating resistor can be converted and the temperature of the heating resistor can be measured by the temperature dependency of the electric resistor. In this way, both the energy input and the temperature change can be controlled and detected by means of the same element.

To this end, FIG. 5 shows an element which will be explained hereinafter. FIG. 5 shows, in section, a metal conductor element 24, to which electrodes 25, 26 are attached. The conductor element 24 is arranged in a fluid flow indicated by the arrow 27, which shows the flow direction. If, as indicated by the arrow 28, a current now passes through the conductor 24, said current will thus also pass through the electrodes 25, 26 at least in part depending on the resistance variables, wherein thermocouples are formed in the transition regions at which a thermoelectric voltage is created by the effect of the current in the region of the material transition. By means of this thermoelectric voltage, which can be measured, the temperature at the conductor 24 can be very precisely determined.

In addition, with suitable material selection in the region of the material transitions between the electrodes 25, 26 and the conductor 24, the Peltier effect can be utilised, which for example entails a production of heat (illustrated by the arrow 29, which represents a heat input) in the region of the current entry from the conductor 24 into an electrode 25, which is a better conductor, whereas a heat absorption occurs in the region in which the current re-enters the conductor 24 from the electrode 25, 26, this being illustrated by the arrow 30. As a result of these effects, a temperature change occurs at the conductor 24 and can be evidenced by means of the above-described heat effect by a change in voltage.

If a fluid flows around the conductor, as in the shown example, said conductor is actively cooled by the fluid. The temperature changes in the event of a certain current flow are thus dependent on the flow rate of the medium flowing around the conductor. By means of a corresponding set of characteristic curves, the through-flow rate of the fluid or the blood can then be determined at a given current through the conductor 24 on the basis of the detected thermoelectric voltage.

The use of a Peltier element, in which both the heating and cooling regions are in contact with blood of a blood pump, can have the advantage that the temperature of the blood as a whole remains largely unchanged by simultaneous, locally distributed heating and cooling.

However, it can also be provided that with use of a Peltier element only a material transition is in contact with the blood and the Peltier element is acted on by changing current directions, such that the specific material transition releases and absorbs heat in alternation. The blood flowing past is thus then likewise heated and cooled in alternation, so that on the whole the blood volume flowing in a patient's body is not changed in respect of temperature.

The through-flow rate of the blood can then be determined with the same element by detection of the thermoelectric voltage. However, it is also conceivable to detect the temperature fluctuations downstream of the blood flow that occur due to the alternating heating and cooling of the blood and to determine the through-flow rate of the blood on the basis of the swings of the temperature towards warmer and cooler temperatures. This can also occur on the basis of a calibration measurement and the use of corresponding characteristic curves.

FIG. 6, in a view in the axial direction, shows a pump tube 2a, which is made of titanium or a titanium alloy and has a wall thickness between 0.1 and 0.7 mm. A heating element 31 is provided at a first point externally on the periphery of the pump tube 2a and is formed as a heating resistor and is fed by means of an external circuit 33. The heating of the heating element 31 results in a local temperature increase of the pump tube 2a in the form of a hot spot, which is indicated by the dashed line 34. The hot spot is cooled from the inner side of the pump tube 2a by the blood flowing past there, wherein the equilibrium temperature of the heating element is dependent on the flow velocity of the blood and allows the flow velocity to be determined.

In order to take into consideration the temperature of the unheated blood when determining the flow velocity, a temperature measurement is taken at the distance a from the heating element 31 by means of the reference temperature sensor 32 on the pump tube 2a. At this point, the pump tube has the temperature of the blood.

On the whole, the invention, by means of the integration of a flowmeter that is based on thermodilution principles, in a pump, allows a space-saving solution, wherein further advantages can be achieved from the integration of the temperature evaluation and of the through-flow rate calculation derived therefrom in the control device for the pump.

1. A pump comprising:
a pump tube; and
a device for determining a through-flow rate, the device for determining the through-flow rate comprising:
a device for supplying or discharging heat energy to or from a fluid conveyed in the pump tube; and
a device for determining a temperature, a temperature change, or a temperature gradient.

2. The pump according to claim 1 further comprising a heating element in direct heat contact with the pump tube, and a device for determining the temperature of the heating element, wherein the device for determining the temperature of the heating element either is integrated in the heating element or is a separate temperature sensor.

3. The pump according to claim 2, wherein a reference temperature sensor is arranged on the pump tube at a distance from the heating element, wherein the distance is at least 2 mm or at least 4 mm, and wherein the heating element is in direct heat contact with the pump tube.

4. The pump according to claim 3, wherein the heating element and the reference temperature sensor are arranged on the pump tube substantially in the same axial position with respect to the longitudinal axis of the pump tube and at a distance from one another in the peripheral direction.

5. The pump according to claim 2, wherein the heating element and the device for determining the temperature of the heating element, the reference temperature sensor, or both are arranged on an outer side of the pump tube.

6. The pump according to claim 2, wherein the pump tube comprises titanium or a titanium alloy and, at least in a portion in which the heating element is arranged, has a wall thickness between 0.1 mm and 1 mm or between 0.1 mm and 0.7 mm.

7. The pump according to claim 1, wherein a device for supplying heat energy to the conveyed fluid includes a drive motor integrated in the pump.

8. The pump according to claim 1, wherein a device for supplying heat energy to the conveyed fluid is formed by one or more active, in particular magnetic bearings integrated in the pump.

9. The pump according to claim 1, wherein a heating or cooling element arranged in a flow of the fluid conveyed in the pump tube, wherein the heating or cooling element includes in the form of a heating wire or a cooling element, or a Peltier element, or both, and wherein the heating or cooling element is arranged in or directly on a housing of the pump.

10. The pump according to claim 1, wherein the device for determining the temperature, the temperature change or the temperature gradient comprises a first and a second temperature sensor, wherein, in a flow of the fluid conveyed in the pump tube, the first temperature sensor is arranged upstream and the second temperature sensor is arranged downstream of the device for supplying or discharging heat energy.

11. The pump according to claim 10, wherein a device for determining the temperature, the temperature change or the temperature gradient comprises a first and a second temperature sensor, wherein, in the flow of the fluid, the first temperature sensor is arranged upstream of a pump rotor and the second temperature sensor is arranged downstream of the pump rotor.

12. The pump according to claim 9, wherein the device for determining the temperature, the temperature change, or the temperature gradient is arranged directly on the heating or cooling element or is integrated therein.

13. The pump according to claim 10, the device for determining the temperature, the temperature change, or the temperature gradient is arranged directly on the heating or cooling element, and wherein the pump further comprises a control device configured to adjust a temperature, a temperature change or a temperature gradient to a target value, wherein the control device is further configured to determine a heating or a cooling power that is to be supplied in order to achieve the target value.

14. The pump according to claim 1, wherein a heat-dissipating region, a cooling region, or both of a temperature element are/is arranged to be in a flow of the fluid, and wherein the pump further comprises one or more temperature sensors for continuous detection of a temperature of the temperature element, of the fluid flowing past, or both, wherein the temperature element comprises a temperature-dependent electrical resistor or a thermocouple.

15. A method for determining a flow rate of a fluid to be conveyed by a pump, the method comprising:
determining the flow rate based on a detected heating or cooling capacity, a temperature, a temperature difference, an electrical motor capacity, and in particular a speed of rotation of the pump and an electric bearing performance.

16. The method of claim 15, wherein the determining the flow rate is further based on a speed of rotation of the pump and an electric bearing performance.

17. The pump according to claim 1, wherein the pump comprises a blood pump.
